(19) United States
(12) Patent Application Publication
(10) Pub. No.: US 2023/0340136 A1
(43) Pub. Date: Oct. 26, 2023

(54) TREATMENT OF CLL

(71) Applicant: NOVARTIS AG, Basel (CH)

(72) Inventors: Liangke GOU, Livingston, NJ (US); Nadia HASSOUNAH, Boston, MA (US); Janghee WOO, Short Hills, NJ (US)

(21) Appl. No.: 18/019,467

(22) PCT Filed: Aug. 2, 2021
(86) PCT No.: PCT/US2021/044115

§ 371 Date: Feb. 2, 2023

Related U.S. Application Data
(60) Provisional application No. 63/060,786, filed on Aug. 4, 2020.

(51) Int. Cl. A61K 31/519; C07K 16/28; A61K 39/395; A61K 31/454; A61P 35/02

(52) U.S. Cl. CPC C07K 16/2878; A61K 31/454; A61K 31/519; A61K 39/3955; A61P 35/02; A61K 2039/545

(57) ABSTRACT

The present invention relates to anti-BAFFR antibodies or binding fragments thereof, alone or in combination with BTK inhibitors, for use in the treatment of CLL. Specifically, the invention relates to a pharmaceutical combination comprising a BTK inhibitor, or a pharmaceutically acceptable salt thereof, and an anti-BAFFR antibody or binding fragment thereof, and their use in the treatment of CLL. The invention also relates to a method for the treatment of CLL that involves administering the combination; and to the use of the combination for the manufacture of a medicament for the treatment of CLL.

### CROSS-REFERENCE TO RELATED APPLICATIONS

This application claims the priority benefit of U.S. Provisional Application No. 63/060,786, filed Aug. 4, 2020, the contents of which are incorporated herein in their entireties by reference thereto.

### SEQUENCE LISTING

The instant application contains a Sequence Listing which has been submitted electronically in ASCII format and is hereby incorporated by reference in its entirety. Said ASCII copy, created on 26 Jul. 2021, is named PAT058936-WO-PCT_SL.txt and is 15,544 bytes in size.

### FIELD OF INVENTION

The present invention relates to anti-BAFFR antibodies or binding fragments thereof, alone or in combination with BTK inhibitors, for use in the treatment of CLL. Specifically, the invention relates to a pharmaceutical combination comprising a BTK inhibitor, or a pharmaceutically acceptable salt thereof, and an anti-BAFFR antibody or binding fragment thereof, and their use in the treatment of CLL. The invention also relates to a method for the treatment of CLL that involves administering the combination; and to the use of the combination for the manufacture of a medicament for the treatment of CLL.

### BACKGROUND

Chronic lymphocytic leukemia (CLL) is the most prevalent adult leukemia in the Western hemisphere. Patients with early stage disease have a greater than 10 year life expectancy. However, patients with more advanced disease have a median survival of only 18 months to 3 years. The development of chemoimmunotherapy regimens combining cytotoxic agents such as alkylating agents and purine nucleoside analogs with monoclonal antibodies such as rituximab have attained overall response (OR) rates of over 90% and CR rates of over 70% in patients with previously untreated CLL, with similar improvement in progression free survival (PFS). Notwithstanding the therapeutic advance represented by chemoimmunotherapy combinations, these treatments are not curative in the majority of cases (Nabhan C and Rosen, ST (2014) Chronic lymphocytic leukemia: a clinical review. JAMA, 312, 2265-2276). Additionally, several features of CLL are predictive of poor response to chemoimmunotherapy treatments as measured by response duration and shortened survival. These include cytogenetic abnormalities resulting in del(17p13.1) and del(11q22.3), or non-mutated immunoglobulin heavy chain variable region genes (IgHV) (Dohner H, Stilgenbauer S, Benner A, et al. (2000) Genomic aberrations and survival in chronic lymphocytic leukemia. N Engl J Med, 343, 1910-1916; Damle RN, Wasil T, Fais F, et al. (1999) Ig V gene mutation status and CD38 expression as novel prognostic indicators in chronic lymphocytic leukemia. Blood, 94, 1840-1847; Chen L, Widhopf G, Huynh L, et al. (2002) Expression of ZAP-70 is associated with increased B-cell receptor signaling in chronic lymphocytic leukemia. Blood, 100, 4609-4614). Patients with del(17p13.1) treated with fludarabine, fludarabine and rituximab, fludarabine plus cyclophosphamide, or fludarabine, cyclophosphamide, and rituximab have a shorter duration of progression-free survival and overall survival compared to patients without this finding (Badoux X, Tam C, Lerner S, et al. (2009) Outcome of First Salvage Therapy in Patients With Chronic Lymphocytic Leukemia Relapsing After First-line Fludarabine, Cyclophosphamide, and Rituximab. Clinical Lymphoma & Myeloma, 9, E39-E40; Tam CS, O’Brien S, Plunkett W, et al. (2014) Long-term results of first salvage treatment in CLL patients treated initially with FCR (fludarabine, cyclophosphamide, rituximab). Blood, 124, 3059-3064). Ibrutinib (PCI-32765, Imbruvica®) is a first-in-class, orally-administered, covalently-binding small molecule inhibitor of BTK (Bruton tyrosine kinase). The chemical name for ibrutinib is 1-[(3R)-3-[4-amino-3-(4-phenoxyphenyl)pyrazolo[3,4-d]pyrimidin-1-yl]piperidin-1-yl]prop-2-en-1-one. BTK is a signaling molecule of the B-cell antigen receptor (BCR) and cytokine receptor pathways. BTK’s role in signaling through the B-cell surface receptors results in activation of pathways necessary for B-cell trafficking, chemotaxis, and adhesion. Ibrutinib is a disease-altering therapy in chronic lymphocytic leukemia and has the advantage of effecting responses in patients with these characteristics associated with poor responses to chemoimmunotherapy. Ibrutinib additionally provides a progression free and overall survival advantage over other standard therapies (Byrd JC, Furman RR, Coutre SE, et al. (2013) Targeting BTK with ibrutinib in relapsed chronic lymphocytic leukemia. N Engl J Med, 369, 32-42; Byrd JC, Furman RR, Coutre S E, et al. (2015) Three-year follow-up of treatment-naive and previously treated patients with CLL and SLL receiving single-agent ibrutinib. Blood, 125, 2497-2506). At the 5-year follow up time from the initial ibrutinib studies, 92% of untreated and 43% of previously heavily treated patients maintain a durable remission (O′Brien SFR, Coutre S, Flinn I, et al (2016) Five-Year Experience With Single-Agent Ibrutinib in Patients With Previously Untreated and Relapsed/Refractory Chronic Lymphocytic Leukemia/Small Lymphocytic Leukemia. Blood (sup), 128, 233). Ibrutinib is approved by the US FDA for all patients with CLL and is widely prescribed as a new standard of care for CLL patients.

However, patients in both untreated and previously treated settings typically have small detectable clonal disease leading to their being classified as minimal residual disease positive (MRD+) CR or PR (partial response). To date, combination studies of BTKi (Bruton’s tyrosine kinase inhibitor) with rituximab, ofatumumab, bendamustine + rituximab, and venetoclax + obinutuzumab have not demonstrated elimination of these residual cells in the majority of patients (Burger JA, Keating MJ, Wierda WG, et al. (2014) Safety and activity of ibrutinib plus rituximab for patients with high-risk chronic lymphocytic leukaemia: a single-arm, phase 2 study. Lancet Oncol, 15, 1090-1099; Jaglowski, SM, Jones, JA, Nagar, V, et al. (2015) Safety and activity of BTK inhibitor ibrutinib combined with ofatumumab in chronic lymphocytic leukemia: a phase 1b/2 study. Blood, 126, 842-850; Chanan-Khan A, Cramer P, Demirkan F, et al. (2016) Ibrutinib combined with bendamustine and rituximab compared with placebo, bendamustine, and rituximab for previously treated chronic lymphocytic leukaemia or small lymphocytic lymphoma (HELlOS): a randomised, double-blind, phase 3 study. Lancet Oncol, 17, 200-211). This long-term persisting MRD+ disease has significant implications for individual patients and the health care system because continuation of ibrutinib is required in this setting.

Additionally, many patients with CLL will stop responding to ibrutinib either by developing transformation to a large-cell lymphoma (Richter’s syndrome) or as progressive CLL, the risk of which increases over time (Maddocks KJ, Ruppert AS, Lozanski G, et al. (2015) Etiology of Ibrutinib Therapy Discontinuation and Outcomes in Patients With Chronic Lymphocytic Leukemia. JAMA Oncol, 1, 80-87). As ibrutinib is dosed indefinitely, the number of patients with progressive CLL during ibrutinib therapy is expected to increase. Furthermore, survival for patients discontinuing ibrutinib due to CLL progression is a short 22.7 months (95% Cl: 13.5-NR) and associated with a clinically aggressive disease phenotype. It is imperative to develop novel treatments for CLL that address lack of MRD (-) CRs and resistance as major limitations of ibrutinib therapy.

Combining BTK inhibitors such as ibrutinib with therapeutic antibodies targeting tumour surface proteins could improve the outome of the treatment. One such surface protein is BAFF receptor (BAFFR). BAFF (B-cell activating factor) is a member of the tumor necrosis factor (TNF) superfamily that supports normal B-cell development and proliferation. BAFFR (also known as BR3, TNFRSF13C, CD268 or BAFF-R) is expressed by B-cells that continue to be active in CLL after treatment with ibrutinib. BAFF-R engagement activates pro-survival activity in B cells by exclusively binding BAFF with high affinity and driving antiapoptotic gene transcription of Bcl-2 family members via NF-kB-inducible kinase-mediated alternative NF-kB signaling.

Antibodies against BAFFR (i.e. “anti-BAFFR antibodies”) are known from e.g. WO 2010/007082 and include antibodies which are characterized by comprising a VH domain with the amino acid sequence of SEQ ID NO: 1 and a VL domain with the amino acid sequence of SEQ ID NO: 2. The antibody MOR6654 is one such antibody (IgG1 kappa). It has the heavy chain amino acid sequence of SEQ ID NO: 9 and the light chain amino acid sequence of SEQ ID NO: 10. This antibody may be expressed from SEQ ID NOs: 14 and 15, preferably in a host cell which lacks fucosyl-transferase, for example in a mammalian cell line with an inactive FUT8 gene (e.g. FUT8^{-/-}), to provide a functional non-fucosylated anti-BAFFR antibody with enhanced ADCC. This antibody is referred to hereafter as MOR6654B or VAY736, or under its international non-proprietary name ianalumab. Alternative ways to produce non-fucosylated antibodies are known in the art.

### BRIEF SUMMARY OF THE DISCLOSURE

In a first aspect the invention relates to an anti-BAFFR antibody or a binding fragment thereof for use in the treatment of CLL in a subject in need thereof, wherein the anti-BAFFR antibody or binding fragment thereof is to be administered at a therapeutically effective dose.

In a second aspect the invention relates to a pharmaceutical combination comprising (i) a BTK inhibitor, and (ii) an anti-BAFFR antibody or a binding fragment thereof, wherein the BTK inhibitor is to be administered at a dose from about 25 mg/day to about 1000 mg/day, and wherein the anti-BAFFR antibody or binding fragment thereof is to be administered at a therapeutically effective dose.

In a third aspect the invention relates to a pharmaceutical combination comprising (i) a BTK inhibitor, and (ii) an anti-BAFFR antibody or a binding fragment thereof, for use in the treatment of CLL in a subject in need thereof, wherein the BTK inhibitor is to be administered at a dose from about 25 mg/day to about 1000 mg/day, and wherein the anti-BAFFR antibody or binding fragment thereof is to be administered at a therapeutically effective dose.

In a fourth aspect the invention relates to the use of the anti-BAFFR antibody or a binding fragment thereof of the first aspect for the manufacture of a medicament.

In a fifth aspect the invention relates to the use of a pharmaceutical combination comprising (i) a BTK inhibitor, and (ii) an anti-BAFFR antibody or a binding fragment thereof for the manufacture of a medicament, wherein the BTK inhibitor is to be administered at a dose from about 25 mg/day to about 1000 mg/day, and wherein the anti-BAFFR antibody or binding fragment thereof is to be administered at a therapeutically effective dose.

In a sixth aspect the invention relates to the use of a pharmaceutical combination comprising (i) a BTK inhibitor, and (ii) an anti-BAFFR antibody or a binding fragment thereof for the manufacture of a medicament for the treatment of CLL, wherein the BTK inhibitor is to be administered at a dose from about 25 mg/day to about 1000 mg/day, and wherein the anti-BAFFR antibody or binding fragment thereof is to be administered at a therapeutically effective dose.

In a seventh aspect the invention relates to a method of for treatment of CLL in a subject in need thereof comprising administering to the subject the anti-BAFFR antibody or a binding fragment thereof of the first aspect, and/or the pharmaceutical combination of the second or third aspect.

### BRIEF DESCRIPTION OF THE DRAWINGS

FIG. 1 shows a schematic presentation of the treatment regimens of Example 2.

FIG. 2 shows percentage change from baseline in blood MRD of the patients treated as in Example 2.

### DEFINITIONS

In order that the present disclosure may be more readily understood, certain terms are first defined. Additional definitions are set forth throughout the detailed description.

As used herein, the term “a”, “an”, “the” and similar terms used in the context of the present disclosure (especially in the context of the claims) are to be construed to cover both the singular and plural unless otherwise indicated herein or clearly contradicted by the context. As such, the terms “a” (or “an”), “one or more”, and “at least one” can be used interchangeably herein.

“And/or” means that each one or both or all of the components or features of a list are possible variants, especially two or more thereof in an alternative or cumulative way.

The term “about” in relation to a numerical value X means, for example, X ± 15%, including all the values within this range.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

In the present disclosure the term “pharmaceutical combination” refers to a non-fixed combination. The term “non-fixed combination” means that the active ingredients, e.g. a BTK inhibitor and an anti-BAFFR antibody are both administered to a patient as separate entities either simultaneously or sequentially with no specific time limits, wherein such administration provides therapeutically effective levels of the two compounds in the body of the patient.

The terms “a combination” or “in combination with” is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

The term “antibody” refers to a protein, e.g., an immunoglobulin chain or fragment thereof, comprising at least one immunoglobulin variable domain sequence. The term “antibody” includes, for example, a monoclonal antibody (including a full length antibody which has an immunoglobulin Fc region). An antibody comprises a full length antibody, or a full length immunoglobulin chain, or an antigen binding or functional fragment of a full length antibody, or a full length immunoglobulin chain. An antibody can also be a multi-specific antibody, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. The term “binding fragment” as used herein refers to a portion of an antibody capable of binding a BAFFR epitope.

The term “Pharmaceutically acceptable salts” can be formed, for example, as acid addition salts, preferably with organic or inorganic acids. Suitable inorganic acids are, for example, halogen acids, such as hydrochloric acid. Suitable organic acids are, e.g., carboxylic acids or sulfonic acids, such as fumaric acid or methanesulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. Any reference to the free compound herein is to be understood as referring also to the corresponding salt, as appropriate and expedient. The salts of the inhibitors, as described herein, are preferably pharmaceutically acceptable salts; suitable counter-ions forming pharmaceutically acceptable salts are known in the field.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “inhibit”, “inhibition” or “inhibiting” refers to the reduction or suppression of a given condition, symptom, or disorder, or disease, or a significant decrease in the baseline activity of a biological activity or process or molecule. For example, inhibition of an activity, e.g., a BTK activity, of at least 5%, 10%, 20%, 30%, 40% or more is included by this term. Thus, inhibition need not be 100%.

As used herein, the term “patient” or “subject” are taken to mean a human. Except when noted, the terms “patient” or “subject” are used herein interchangeably.

As used herein, a subject is “in need of” a treatment if such subject would benefit biologically, medically or in quality of life from such treatment.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition. The benefit to a patient to be treated is either statistically significant or at least perceptible to the patient or to the physician. However, it will be appreciated that when a medicament is administered to a patient to treat a disease, the outcome may not always be an effective treatment.

The terms “drug”, “active substance”, “active ingredient”, “pharmaceutically active ingredient”, “active agent”, “therapeutic agent” or “agent” are to be understood as meaning a compound in free form or in the form of a pharmaceutically acceptable salt.

By the term “effective amount” or “therapeutically effective amount” or “pharmaceutically effective amount”, is meant the amount or quantity of active agent that is sufficient to elicit the required or desired response, or in other words, the amount that is sufficient to elicit an appreciable biological response when administered to a subject. Said amount preferably relates to an amount that is therapeutically or in a broader sense also prophylactically effective against the progression of a disease or disorder as disclosed herein. It is understood that an “effective amount” or a “therapeutically effective amount” can vary from subject to subject, due to variation in metabolism of the drug, age, weight, general condition of the subject, the condition being treated, the severity of the condition being treated, and the judgment of the prescribing physician.

The term “anti-BAFFR antibody or binding fragment thereof” as used herein refers to an antibody, or binding fragment thereof, which comprises a BAFFR binding domain. The binding of the antibody (or binding fragment thereof) to BAFFR inhibits the binding of BAFFR to BAFF and thereby reduces the formation of BAFF/BAFFR complexes, and/or reduce the activation of BAFFR. Suitably, the anti-BAFFR antibody or binding fragment thereof may reduce the formation of BAFF/BAFFR complexes and/or reduce the activation of BAFFR by at least 30%, at least 40%, at least 50%, at least 60%, at least 70%, at least 80%, at least 90%, at least 95%, at least 96%, at least 97%, at least 98%, at least 99%, or more as compared to a suitable control (for example a sample without the presence of an anti-BAFFR antibody or binding fragment thereof). Additionally or alternatively, an anti-BAFFR antibody or binding thereof may dissociate preformed BAFF/BAFFR complexes. In a suitable embodiment antibody or binding fragment thereof may dissociate at least 30%, at least 40%, at least 50%, at least 60%, at least 70%, at least 80%, at least 90%, at least 95%, at least 96%, at least 97%, at least 98%, at least 99%, or more of preformed BAFF/BAFFR complexes. As before, this property may be compared to a suitable control (for example a sample without the presence of an anti-BAFFR antibody or binding fragment thereof).

### DETAILED DESCRIPTION

The present invention is based on the inventors’ surprising finding that an anti-BAFFR antibody or binding fragment thereof, or a pharmaceutical combination comprising a BTK inhibitor and an anti-BAFFR antibody or binding fragment thereof, particularly when administered at a specific dosage regimen as disclosed herein, has an exceptional efficacy, safety and tolerability for treatment of CLL. Clinical studies with the anti-BAFFR antibody ianalumab (VAY736) reported in the Examples of the subject application support the use of anti-BAFFR antibodies and binding fragments thereof as an efficacious treatment of CLL.

Therefore, in a first aspect, the invention relates to an anti-BAFFR antibody or a binding fragment thereof for use in the treatment of CLL in a subject in need thereof, wherein the anti-BAFFR antibody or binding fragment thereof is to be administered at a therapeutically effective dose.

In one embodiment, the anti-BAFFR antibody or binding fragment thereof is administered at a dose of about 0.1 mg/kg to about 10 mg/kg, preferably from about 0.3 mg/kg to about 9 mg/kg, more preferably from about 1 mg/kg to about 6 mg/kg. In a preferred embodiment, the anti-BAFFR antibody or binding fragment thereof is administered at a dose of about 3 mg/kg. In another preferred embodiment, the anti-BAFFR antibody or binding fragment thereof is administered at a dose of about 9 mg/kg.

In one embodiment, the anti-BAFFR antibody or binding fragment thereof comprises a heavy chain variable region comprising three CDRs consisting of SEQ ID NO: 3, SEQ ID NO: 4, and SEQ ID NO: 5, respectively and a light chain variable region comprising three CDRs consisting of SEQ ID NO: 6, SEQ ID NO: 7, and SEQ ID NO: 8, respectively. In a preferred embodiment, the anti-BAFFR antibody or binding fragment thereof comprises a heavy chain variable region consisting of the sequence SEQ ID NO: 1 and a light chain variable region consisting of the sequence SEQ ID NO: 2. In a more preferred embodiment, the anti-BAFFR antibody or binding fragment thereof is ianalumab or binding fragment thereof.

In one embodiment, ianalumab or binding fragment thereof is administered at a dose of about 0.1 mg/kg, 0.3 mg/kg, 1 mg/kg, 3 mg/kg, 6 mg/kg or 9 mg/kg. In a preferred embodiment, ianalumab or binding fragment thereof is administered at a dose of about 3 mg/kg. In another preferred embodiment, ianalumab or binding fragment thereof is administered at a dose of about 9 mg/kg.

In one embodiment, the anti-BAFFR antibody or a binding fragment thereof is administered to a subject in need thereof every four (4) weeks (q4w) (+/- 3 days), or every two (2) weeks (q2w) (+/-3 days). In a preferred embodiment, the anti-BAFFR antibody or a binding fragment thereof is administered every two (2) weeks (q2w) (+/- 3 days). In a preferred embodiment, ianalumab or binding fragment thereof is administered every two (2) weeks (q2w) (+/- 3 days). In another preferred embodiment, the anti-BAFFR antibody or a binding fragment thereof is administered every four (4) weeks (q4w) (+/- 3 days). In another preferred embodiment, ianalumab or binding fragment thereof is administered every four (4) weeks (q4w) (+/- 3 days).

In a preferred embodiment, ianalumab or binding fragment thereof is administered every two (2) weeks (q2w) (+/- 3 days) at a dose of about 3 mg/kg.

In another preferred embodiment, ianalumab or binding fragment thereof is administered every four (4) weeks (q4w) (+/- 3 days) at a dose of about 9 mg/kg.

The antibody or binding fragment thereof can be administered by a variety of methods known in the art, although for many therapeutic applications, the preferred route/mode of administration is intravenous injection or infusion. For example, the antibody or binding fragment thereof can be administered by intravenous infusion at a rate of more than about 5 mg/min, e.g., 10-40 mg/min, and typically greater than or equal to 20 mg/min to reach a dose of about 150 to 400 mg per infusion. For intravenous injection or infusion, therapeutic compositions typically should be sterile and stable under the conditions of manufacture and storage. The composition can be formulated as a solution, microemulsion, dispersion, liposome, or other ordered structure suitable for the antibody and its concentration. Sterile injectable solutions can be prepared by incorporating the active compound (i.e., antibody or binding fragment thereof) in the required amount in an appropriate solvent with one or a combination of ingredients as required, followed by filtered sterilization. Generally, dispersions are prepared by incorporating the active compound into a sterile vehicle that contains a basic dispersion medium and the required other ingredients. It would be understood that the route and/or mode of administration will vary depending upon the desired results. For example, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art (e.g., Sustained and Controlled Release Drug Delivery Systems, J. R. Robinson, ed., Marcel Dekker, Inc., New York, 1978). In a preferred embodiment, the anti-BAFFR antibody or binding fragment thereof, e.g. ianalumab or binding fragment thereof, is administered intravenously to a subject in need thereof.

In a second aspect, the invention relates to a pharmaceutical combination comprising (i) a BTK inhibitor, and (ii) an anti-BAFFR antibody or a binding fragment thereof, wherein the BTK inhibitor is to be administered at a dose from about 25 mg/day to about 1000 mg/day, and wherein the anti-BAFFR antibody or binding fragment thereof is to be administered at a therapeutically effective dose.

In one embodiment, the anti-BAFFR antibody or binding fragment thereof is administered at a dose of about 0.1 mg/kg to about 10 mg/kg, preferably from about 0.3 mg/kg to about 9 mg/kg, more preferably from about 1 mg/kg to about 6 mg/kg. In a preferred embodiment, the anti-BAFFR antibody or binding fragment thereof is administered at a dose of about 3 mg/kg. In another preferred embodiment, the anti-BAFFR antibody or binding fragment thereof is administered at a dose of about 9 mg/kg.

In one embodiment, the anti-BAFFR antibody or binding fragment thereof comprises a heavy chain variable region comprising three CDRs consisting of SEQ ID NO: 3, SEQ ID NO: 4, and SEQ ID NO: 5, respectively and a light chain variable region comprising three CDRs consisting of SEQ ID NO: 6, SEQ ID NO: 7, and SEQ ID NO: 8, respectively. In a preferred embodiment, the anti-BAFFR antibody or binding fragment thereof comprises a heavy chain variable region consisting of the sequence SEQ ID NO: 1 and a light chain variable region consisting of the sequence SEQ ID NO: 2. In a more preferred embodiment, the anti-BAFFR antibody or binding fragment thereof is ianalumab or binding fragment thereof.

In one embodiment, ianalumab or binding fragment thereof is administered at a dose of about 0.1 mg/kg, 0.3 mg/kg, 1 mg/kg, 3 mg/kg, 6 mg/kg or 9 mg/kg. In a preferred embodiment, ianalumab or binding fragment thereof is administered at a dose of about 3 mg/kg. In another preferred embodiment, ianalumab or binding fragment thereof is administered at a dose of about 9 mg/kg.

In one embodiment, the anti-BAFFR antibody or a binding fragment thereof is administered to a subject in need thereof every four (4) weeks (q4w) (+/- 3 days), or every two (2) weeks (q2w) (+/-3 days). In a preferred embodiment, the anti-BAFFR antibody or a binding fragment thereof is administered every two (2) weeks (q2w) (+/- 3 days). In a preferred embodiment, ianalumab or binding fragment thereof is administered every two (2) weeks (q2w) (+/- 3 days). In another preferred embodiment, the anti-BAFFR antibody or a binding fragment thereof is administered every four (4) weeks (q4w) (+/- 3 days). In another preferred embodiment, ianalumab or binding fragment thereof is administered every four (4) weeks (q4w) (+/- 3 days).

In a preferred embodiment, ianalumab or binding fragment thereof is administered every two (2) weeks (q2w) (+/- 3 days) at a dose of about 3 mg/kg.

In another preferred embodiment, ianalumab or binding fragment thereof is administered every four (4) weeks (q4w) (+/- 3 days) at a dose of about 9 mg/kg.

In a preferred embodiment, the anti-BAFFR antibody or binding fragment thereof, e.g. ianalumab or binding fragment thereof, is administered intravenously to a subject in need thereof.

Several BTK inhibitors are available or under development for therapeutic use and known in the art; an overview of such BTK inhibitors is provided e.g. by Bond and Woyach, 2019 (doi: 10.1007/s11899-019-00512-0) and Feng et al., 2019 (doi: 10.1080/13543776.2019.1594777), both of which are hereby incorporated by reference. In one embodiment, the BTK inhibitor is ibrutinib, acalabrutinib, zanubrutinib, spebrutinib, olmutinib, tirabrutinib, evobrutinib, fenebrutinib, vecabrutinib, BMS-986142, PRN1008, ABBV-105, TAS5315, APQ531, M7583, SHR1459, CT-1530, TG-1701, BIIB068, SAR442168, AC0058, DTRMWXHS-12, GDC-0834, RN-486, or a pharmaceutically acceptable salt thereof. In a preferred embodiment, the BTK inhibitor is ibrutinib or a pharmaceutically acceptable salt thereof. Preferrably, ibrutinib or a pharmaceutically acceptable salt thereof is administered at a daily dose of about 140 mg to about 840 mg, or about 280 mg to about 700 mg, preferably about 420 mg.

In a preferred embodiment, the BTK inhibitor is ibrutinib or a pharmaceutically acceptable salt thereof and the anti-BAFFR antibody is ianalumab or a binding fragment thereof, wherein the ianalumab or binding fragment thereof is administered every two (2) weeks (q2w) (+/- 3 days) at a dose of about 3 mg/kg.

In another preferred embodiment, the BTK inhibitor is ibrutinib or a pharmaceutically acceptable salt thereof and the anti-BAFFR antibody is ianalumab or a binding fragment thereof, wherein the ianalumab or binding fragment thereof is administered every four (4) weeks (q4w) (+/- 3 days) at a dose of about 9 mg/kg.

In a third aspect the invention relates to a pharmaceutical combination comprising (i) a BTK inhibitor, and (ii) an anti-BAFFR antibody or a binding fragment thereof, for use in the treatment of CLL in a subject in need thereof, wherein the BTK inhibitor is to be administered at a dose from about 25 mg/day to about 1000 mg/day, and wherein the anti-BAFFR antibody or binding fragment thereof is to be administered at a therapeutically effective dose.

In one embodiment, the anti-BAFFR antibody or binding fragment thereof is administered at least once per cycle. Each cycle is 28 days.

In one embodiment, the anti-BAFFR antibody or a binding fragment thereof is administered to a subject in need thereof every four (4) weeks (q4w) (+/- 3 days), or every two (2) weeks (q2w) (+/-3 days). In a preferred embodiment, the anti-BAFFR antibody or a binding fragment thereof is administered every two (2) weeks (q2w) (+/- 3 days). In a preferred embodiment, ianalumab or binding fragment thereof is administered every two (2) weeks (q2w) (+/- 3 days). In another preferred embodiment, the anti-BAFFR antibody or a binding fragment thereof is administered every four (4) weeks (q4w) (+/- 3 days). In another preferred embodiment, ianalumab or binding fragment thereof is administered every four (4) weeks (q4w) (+/- 3 days).

In one embodiment, the anti-BAFFR antibody or a binding fragment thereof is administered for 6 cycles only.

In one embodiment, the pharmaceutical combination is administered for at least 6 cycles.

In one embodiment, the BTK inhibitor is administered for at least 8 cycles.

In one embodiment, the pharmaceutical combination is administered for 6 cycles, followed by administration of the BTK inhibitor for 2 cycles. In a preferred embodiment, the anti-BAFFR antibody or a binding fragment thereof is ianalumab or binding fragment thereof. In a preferred embodiment, the BTK inhibitor is ibrutinib or a pharmaceutically acceptable salt thereof.

In one embodiment, the anti-BAFFR antibody or binding fragment thereof comprises a heavy chain variable region comprising three CDRs consisting of SEQ ID NO: 3, SEQ ID NO: 4, and SEQ ID NO: 5, respectively and a light chain variable region comprising three CDRs consisting of SEQ ID NO: 6, SEQ ID NO: 7, and SEQ ID NO: 8, respectively. In a preferred embodiment, the anti-BAFFR antibody or binding fragment thereof comprises a heavy chain variable region consisting of the sequence SEQ ID NO: 1 and a light chain variable region consisting of the sequence SEQ ID NO: 2. In a more preferred embodiment, the anti-BAFFR antibody or binding fragment thereof is ianalumab or binding fragment thereof.

In one embodiment, the anti-BAFFR antibody or binding fragment thereof is administered at a dose of about 0.1 mg/kg to about 10 mg/kg, preferably from about 0.3 mg/kg to about 9 mg/kg, more preferably from about 1 mg/kg to about 6 mg/kg. In a preferred embodiment, the anti-BAFFR antibody or binding fragment thereof is administered at a dose of about 3 mg/kg. In another preferred embodiment, the anti-BAFFR antibody or binding fragment thereof is administered at a dose of about 9 mg/kg.

In one embodiment, ianalumab or binding fragment thereof is administered at a dose of about 0.1 mg/kg, 0.3 mg/kg, 1 mg/kg, 3 mg/kg, 6 mg/kg or 9 mg/kg. In a preferred embodiment, ianalumab or binding fragment thereof is administered at a dose of about 3 mg/kg. In another preferred embodiment, ianalumab or binding fragment thereof is administered at a dose of about 9 mg/kg.

In a preferred embodiment of the invention disclosed herein, the BTK inhibitor is ibrutinib or a pharmaceutically acceptable salt thereof. Ibrutinib is an orally bioavailable and irreversible and highly potent small molecule BTK inhibitor. It is an irreversible inhibitor that covalently acted on Cys481 in the ATP binding site of BTK with an IC₅₀ value of 0.5 nM. In a more preferred embodiment, ibrutinib or a pharmaceutically acceptable salt thereof is administered at a daily dose of about 140 mg to about 840 mg, or about 280 mg to about 700 mg, preferably about 420 mg.

In another preferred embodiment, the BTK inhibitor is acalabrutinib (ACP-196) or a pharmaceutically acceptable salt thereof. As an analog of ibrutinib and a second-generation BTK inhibitor, acalabrutinib is reported to have better selectivity and safety than the first-generation ibrutinib and improved off-target effect (Barf T, CoveyT, IzumiR, et al. Acalabrutinib (ACP-196): A covalent bruton tyrosine kinase inhibitor with a differentiated selectivity and in vivo potency profile. Bioorg J Pharmacol Exp Ther. 2017;363:240-252). Preferably, acalabrutinib or a pharmaceutically acceptable salt thereof is administered at a daily dose of about 200 mg (e.g. 100 mg bid).

In another preferred embodiment, the BTK inhibitor is zanubrutinib (BGB-3111) or a pharmaceutically acceptable salt thereof. Preferably, zanubrutinib or a pharmaceutically acceptable salt thereof is administered at a daily dose of about 320 mg (e.g. 160 mg bid).

In another preferred embodiment, the BTK inhibitor is spebrutinib (CC-292/AVL-292), which is a covalent, orally bioavailable BTK inhibitor with an IC50 below 0.5 nM, or a pharmaceutically acceptable salt thereof. Preferably, spebrutinib or a pharmaceutically acceptable salt thereof is administered at a daily dose of about 125 mg, about 250 mg, about 400 mg, about 625 mg, about 750 mg, or about 1000 mg.

In another preferred embodiment, the BTK inhibitor is tirabrutinib (ONO/GS-4059), which is a highly selective and an irreversible BTK inhibitor, inhibiting BTK with an IC50 value of 2.2 nM, or a pharmaceutically acceptable salt thereof, e.g. tirabrutinib hydrochloride. Preferably, tirabrutinib or a pharmaceutically acceptable salt thereof is administered at a daily dose of about 80 mg, about 160 mg, about 320 mg, about 480 mg, or about 600 mg.

In another preferred embodiment, the BTK inhibitor is fenebrutinib (GDC-0853), which is uniquely reversible and selective BTK inhibitor active against ibrutinib-resistant BTK^{C481S} mutantion, or a pharmaceutically acceptable salt thereof. Preferably, fenebrutinib or a pharmaceutically acceptable salt thereof is administered at a daily dose of about 100 mg, 200 mg, or 400 mg.

In another preferred embodiment, the BTK inhibitor is vecabrutinib (SNS-062), which is a potent, noncovalent BTK and ITK inhibitor with a K_{d} value of 0.3 nM, or a pharmaceutically acceptable salt thereof. Preferably, vecabrutinib or a pharmaceutically acceptable salt thereof is administered at a daily dose of about 25 mg, 50 mg, 100 mg, 200 mg, 300 mg, or 400 mg.

In another preferred embodiment, the BTK inhibitor is olmutinib or a pharmaceutically acceptable salt thereof. Preferably, olmutinib or a pharmaceutically acceptable salt thereof is administered at a daily dose of about 800 mg.

In some embodiments, the BTK inhibitor is BMS-986142 (6-Fluoro-5-(R)-(3-(S)-(8-Fluoro-1-Methyl-2,4-Dioxo-1,2-Dihydroquinazolin-3(4h)-YI)-2-Methylphenyl)-2-(S)-(2-Hydroxypropan-2-Yl)-2,3,4,9-Tetrahydro-1h-Carbazole-8-Carboxamide), evobrutinib, PRN1008 ((R,E)-2-(3-(4-amino-3-(2-fluoro-4-phenoxyphenyl)-1H-pyrazolo[3,4-d]pyrimidin-1-yl)piperidine-1-carbonyl)-4-methyl-4-(4-(oxetan-3-yl)piperazin-1-yl)pent-2-enenitrile), ABBV-105 (described in WO2014210255, APQ531, M7583, SHR1459, CT-1530, TG-1701, BIIB068, SAR442168, AC0058, DTRMWXHS-12, GDC-0834, RN-486, or a pharmaceutically acceptable salt thereof.

In one embodiment, CLL is relapsed/refractory (R/R CLL).

In one embodiment, the subject has a mutation conferring resistance to the BTK inhibitor. Several such mutations, the underlying molecular mechanisms, and the methods for detection of such mutations are known in the art and reported e.g. by Ahn et al., 2017 (doi: 10.1182/blood-2016-06-719294), Pula et al., 2019 (doi: 10.3390/cancers11121834), Zhou et al., 2020 (doi: 10.2147/OTT.S249586), George et al., 2020 (doi: 10.3390/cancers12051328), and Woyach et al., 2018 (doi: 10.1056/NEJMoa1400029), all of which are hereby incorporated by reference.

In one embodiment, the resistance mutation is in BTK, PLCG2 and/or TP53 genes.

In one embodiment, the resistance mutation is at ≥1% variant allele frequency or at <1% with two separate measurements at least 4 weeks apart with increasing variant allele frequency.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

Abbreviations

| | |
|---|---|
| ADCC | Antibody Dependent Cellular Cytotoxicity |
| AE | Adverse Event |
| ANC | Absolute Neutrophil Count |
| BAFF | B-cell Activating Factor |
| BAFF-R | B-cell Activating Factor Receptor |
| sBAFF | soluble BAFF |
| BLRM | Bayesian Logistic Regression Model |
| BOR | Best Overall Response |
| BTK | Bruton’s Tyrosine Kinase |
| BTKi | Bruton’s Tyrosine Kinase inhibitor |
| CI | Confidence Interval |
| CLL | Chronic Lymphocytic Leukemia |
| CNS | Central Nervous System |
| CR | Complete Response |
| CT | Computed Tomography |
| DLT | Dose Limiting Toxicity |
| ECOG | Eastern Cooperative Oncology Group |
| eCRF | Electronic Case Report Form |
| EWOC | Escalation With Overdose Control |
| FAS | Full Analysis Set |
| FDA | Food and Drug Administration |
| G-CSF | Granulocyte Colony Stimulating Factor |
| GVHD | Graft vs. Host Disease |
| Hgb | Hemoglobin |
| HIV | Human Immunodeficiency Virus |
| Ig | Immunogenicity |
| IgA | Immunoglobulin A |
| IgG | Immunoglobulin G |
| IgG1 | Immunoglobulin G1 |
| IgM | Immunoglobulin M |
| IUD | Intrauterine Device |
| IUS | Intrauterine System |
| i.v. | Intravenous(ly) |
| IWCLL | International Working Group - CLL |
| mAb | Monoclonal antibody |
| MAP | Meta-Analytic-Predictive |
| MRD | Minimal Residual Disease |
| MRI | Magnetic Resonance Imaging |
| MTD | Maximum Tolerated Dose |
| ΝF-κΒ | Nuclear Factor-Kappa Beta |
| NK | Natural Killer |
| NYHA | New York Heart Association |
| ORR | Overall Response Rate |
| PD | Pharmacodynamics |
| PFS | Progression-Free Survival |
| PK | Pharmacokinetics |
| PR | Partial Response |
| PR-L | PR with lymphocytosis |
| Q2W | Once Every Two Weeks |
| Q4W | Once Every Four Weeks |
| RA | Rheumatoid Arthritis |
| RD | Recommended dose |
| RO | Receptor Occupancy |
| SAE | Serious Adverse Event |
| SD | Stable Disease |
| TTP | Time to Progression |
| ULN | Upper Limit of Normal |
| WHO | World Health Organization |

### EXAMPLES

### EXAMPLE 1: Preparing Anti-BAFFR Antibodies

To enable a person skilled in the art to practice the invention, the amino acid and nucleotide sequences of ianalumab are provided below.

Antibody ianalumab (MOR6654, or VAY736) binds specifically to BAFFR and is also described in international application published as WO2010/007082. It is a human IgG1 kappa antibody obtained via phage display. Its heavy and light chains consist of SEQ ID NOs: 9 and 10, respectively. The Tables 1 and 2 below summarize the sequence characteristics of ianalumab.

TABLE 1

Brief description of the sequences listed in the sequence listing of Table 2.

| SEQ ID NO: | Description of the sequence |
|---|---|
| 1 | Amino acid sequence of the variable region (V_{H}) of the heavy chain of VAY736 |
| 2 | Amino acid sequence of the variable region (V_{L}) of the light chain of VAY736 |
| 3 | Amino acid sequence of HCDR1 of VAY736 |
| 4 | Amino acid sequence of HCDR2 of VAY736 |
| 5 | Amino acid sequence of HCDR3 of VAY736 |
| 6 | Amino acid sequence of LCDR1 of VAY736 |
| 7 | Amino acid sequence of LCDR2 of VAY736 |
| 8 | Amino acid sequence of LCDR3 of VAY736 |
| 9 | Amino acid sequence of the full length heavy chain of VAY736 |
| 10 | Amino acid sequence of the full length light chain of VAY736 |
| 11 | Nucleotide sequence encoding SEQ ID NO:1 |
| 12 | Nucleotide sequence encoding SEQ ID NO:2 |
| 13 | Human BAFFR amino acid sequence |
| 14 | Full length nucleotide sequence (including leader sequence and constant part) of MOR6654 heavy chain; nt 1-57 = leader; nt 58-429 = VH; nt 430-1419 = constant region (hlgG1) |
| 15 | Full length nucleotide sequence (including leader sequence and constant part) of MOR6654 light chain; nt 1-60 = leader; nt 61-384 = VL; nt 385-705 = constant region (hkappa) |

TABLE 2

Sequence listing

| | |
|---|---|
| SEQ ID NO: | Amino acid or Nucleotide Sequence |
| 1 | QVQLQQSGPGLVKPSQTLSLTCAISGDSVSSNSAAWGWIRQSPGRGLEWLG RIYYRSKWYNSYAVSVKSRITINPDTSKNQFSLQLNSVTPEDTAVYYCARYD WVPKIGVFDSWGQGTLVTVSS |
| 2 | DIVLTQSPATLSLSPGERATLSCRASQFISSSYLSWYQQKPGQAPRLLIYGSS SRATGVPARFSGSGSGTDFTLTISSLEPEDFAVYYCQQLYSSPMTFGQGTKV EIK |
| 3 | GDSVSSNSAAWG |
| 4 | RIYYRSKWYNSYAVSVKS |
| 5 | YDWVPKIGVFDS |
| 6 | RASQFISSSYLS |
| 7 | GSSSRAT |
| 8 | QQLYSSPMT |
| 9 | QVQLQQSGPGLVKPSQTLSLTCAISGDSVSSNSAAWGWIRQSPGRGLEWLG RIYYRSKWYNSYAVSVKSRITINPDTSKNQFSLQLNSVTPEDTAVYYCARYDW VPKIGVFDSWGQGTLVTVSSASTKGPSVFPLAPSSKSTSGGTAALGCLVKDY FPEPVTVSWNSGALTSGVHTFPAVLQSSGLYSLSSVVTVPSSSLGTQTYICNV NHKPSNTKVDKRVEPKSCDKTHTCPPCPAPELLGGPSVFLFPPKPKDTLMIS RTPEVTCVVVDVSHEDPEVKFNWYVDGVEVHNAKTKPREEQYNSTYRVVSV LTVLHQDWLNGKEYKCKVSN KALPAPI EKTISKAKGQPREPQVYTLPPSREE MTKNQVSLTCLVKGFYPSDIAVEWESNGQPENNYKTTPPVLDSDGSFFLYSK LTVDKSRWQQGNVFSCSVMHEALHNHYTQKSLSLSPGK |
| 10 | DIVLTQSPATLSLSPGERATLSCRASQFISSSYLSWYQQKPGQAPRLLIYGSS SRATGVPARFSGSGSGTDFTLTISSLEPEDFAVYYCQQLYSSPMTFGQGTKV EIKRTVAAPSVFIFPPSDEQLKSGTASVVCLLNNFYPREAKVQWKVDNALQSG NSQESVTEQDSKDSTYSLSSTLTLSKADYEKHKVYACEVTHQGLSSPVTKSF NRGEC |
| 11 | CAGGTGCAGCTGCAGCAGAGCGGCCCAGGCCTGGTCAAGCCCTCTCAGA CCCTGTCACTGACCTGCGCCATTTCAGGCGACAGCGTGAGCAGCAACAG CGCCGCCTGGGGCTGGATCAGGCAGAGCCCCGGTAGGGGCCTGGAATG GCTGGGCAGGATCTACTACAGGTCCAAGTGGTACAACAGCTACGCCGTG AGCGTGAAGAGCAGGATCACCATCAACCCTGACACCAGCAAGAACCAGTT CTCACTGCAGCTCAACAGCGTGACCCCCGAGGACACCGCCGTGTACTAC TGCGCCAGATACGACTGGGTGCCCAAGATCGGCGTGTTCGACAGCTGGG GCCAGGGCACCCTGGTGACCGTGTCAAGC |
| 12 | GATATCGTGCTGACACAGAGCCCCGCCACCCTGAGCCTGAGCCCAGGCG AGAGGGCCACCCTGTCCTGCAGGGCCAGCCAGTTTATCAGCAGCAGCTA CCTGTCCTGGTATCAGCAGAAGCCCGGCCAGGCCCCTAGACTGCTGATC TACGGCAGCTCCTCTCGGGCCACCGGCGTGCCCGCCAGGTTCAGCGGC AGCGGCTCCGGCACCGACTTCACCCTGACAATCAGCAGCCTGGAGCCCG AGGACTTCGCCGTGTACTACTGCCAGCAGCTGTACAGCTCACCCATGACC TTCGGCCAGGGCACCAAGGTGGAGATCAAG |
| 13 | MRRGPRSLRGRDAPAPTPCVPAECFDLLVRHCVACGLLRTPRPKPAGASSP APRTALQPQESVGAGAGEAALPLPGLLFGAPALLGLALVLALVLVGLVSWRR |
| | RQRRLRGASSAEAPDGDKDAPEPLDKVIILSPGISDATAPAWPPPGEDPGTT PPGHSVPVPATELGSTELVTTKTAGPEQQ |
| 14 | ATGGCCTGGGTGTGGACCCTGCCCTTCCTGATGGCCGCTGCCCAGTCAG TGCAGGCCCAGGTGCAGCTGCAGCAGAGCGGCCCAGGCCTGGTCAAGC CCTCTCAGACCCTGTCACTGACCTGCGCCATTTCAGGCGACAGCGTGAG CAGCAACAGCGCCGCCTGGGGCTGGATCAGGCAGAGCCCCGGTAGGGG CCTGGAATGGCTGGGCAGGATCTACTACAGGTCCAAGTGGTACAACAGCT ACGCCGTGAGCGTGAAGAGCAGGATCACCATCAACCCTGACACCAGCAA GAACCAGTTCTCACTGCAGCTCAACAGCGTGACCCCCGAGGACACCGCC GTGTACTACTGCGCCAGATACGACTGGGTGCCCAAGATCGGCGTGTTCG ACAGCTGGGGCCAGGGCACCCTGGTGACCGTGTCAAGCGCCAGCACCAA GGGCCCCAGCGTGTTCCCCCTGGCCCCCAGCAGCAAGAGCACCAGCGG CGGCACAGCCGCCCTGGGCTGCCTGGTGAAGGACTACTTCCCCGAGCCC GTGACCGTGTCCTGGAACAGCGGAGCCCTGACCTCCGGCGTGCACACCT TCCCCGCCGTGCTGCAGAGCAGCGGCCTGTACAGCCTGTCCAGCGTGGT GACAGTGCCCAGCAGCAGCCTGGGCACCCAGACCTACATCTGCAACGTG AACCACAAGCCCAGCAACACCAAGGTGGACAAGAGAGTGGAGCCCAAGA GCTGCGACAAGACCCACACCTGCCCCCCCTGCCCAGCCCCAGAGCTGCT GGGCGGACCCTCCGTGTTCCTGTTCCCCCCCAAGCCCAAGGACACCCTG ATGATCAGCAGGACCCCCGAGGTGACCTGCGTGGTGGTGGACGTGAGCC ACGAGGACCCAGAGGTGAAGTTCAACTGGTACGTGGACGGCGTGGAGGT GCACAACGCCAAGACCAAGCCCAGAGAGGAGCAGTACAACAGCACCTAC AGGGTGGTGTCCGTGCTGACCGTGCTGCACCAGGACTGGCTGAACGGCA AGGAATACAAGTGCAAGGTCTCCAACAAGGCCCTGCCAGCCCCCATCGA AAAGACCATCAGCAAGGCCAAGGGCCAGCCACGGGAGCCCCAGGTGTAC ACCCTGCCCCCCTCCCGGGAGGAGATGACCAAGAACCAGGTGTCCCTGA CCTGTCTGGTGAAGGGCTTCTACCCCAGCGACATCGCCGTGGAGTGGGA GAGCAACGGCCAGCCCGAGAACAACTACAAGACCACCCCCCCAGTGCTG GACAGCGACGGCAGCTTCTTCCTGTACAGCAAGCTGACCGTGGACAAGT CCAGGTGGCAGCAGGGCAACGTGTTCAGCTGCAGCGTGATGCACGAGGC CCTGCACAACCACTACACCCAGAAGAGCCTGAGCCTGTCCCCCGGCAAG |
| 15 | ATGAGCGTGCTGACCCAGGTGCTGGCTCTGCTGCTGCTGTGGCTGACCG GCACCAGATGCGATATCGTGCTGACACAGAGCCCCGCCACCCTGAGCCT GAGCCCAGGCGAGAGGGCCACCCTGTCCTGCAGGGCCAGCCAGTTTATC AGCAGCAGCTACCTGTCCTGGTATCAGCAGAAGCCCGGCCAGGCCCCTA GACTGCTGATCTACGGCAGCTCCTCTCGGGCCACCGGCGTGCCCGCCAG |
| | GTTCAGCGGCAGCGGCTCCGGCACCGACTTCACCCTGACAATCAGCAGC CTGGAGCCCGAGGACTTCGCCGTGTACTACTGCCAGCAGCTGTACAGCT CACCCATGACCTTCGGCCAGGGCACCAAGGTGGAGATCAAGCGTACGGT GGCCGCTCCCAGCGTGTTCATCTTCCCCCCCAGCGACGAGCAGCTGAAG AGCGGCACCGCCAGCGTGGTGTGCCTGCTGAACAACTTCTACCCCCGGG AGGCCAAGGTGCAGTGGAAGGTGGACAACGCCCTGCAGAGCGGCAACA GCCAGGAGAGCGTCACCGAGCAGGACAGCAAGGACTCCACCTACAGCCT GAGCAGCACCCTGACCCTGAGCAAGGCCGACTACGAGAAGCATAAGGTG TACGCCTGCGAGGTGACCCACCAGGGCCTGTCCAGCCCCGTGACCAAGA GCTTCAACAGGGGCGAGTGC |

### EXAMPLE 2: Phase lb Open-label Study of VAY736 and Ibrutinib in Patients with Chronic Lymphocytic Leukemia (CLL) on Ibrutinib Therapy

### 2.1 Methods

### 2.1.1 Study Description and Design

The purpose of this study (CVAY736Y2102) is to determine the safe and tolerable dose of VAY736 for use in combination with ibrutinib and explore preliminary efficacy of the combination. After the safe and tolerable dose of VAY736 is determined in dose escalation, two expansion arms will enroll CLL patients currently taking ibrutinib who have either failed to achieve a CR after >1 year of treatment or who have developed a mutation known to confer molecular resistance to ibrutinib and predict relapse. The dose expansion part of the study will also include patients who have received ibrutinib either alone or in combination (or have received ibrutinib continuously with multiple sequential combination partners) as first-line therapy and have either failed to achieve a complete response after 1 year of therapy or have developed a resistance mutation to ibrutinib. The purpose of the expansion arms is to gather preliminary efficacy data in these specific groups and will test whether addition of VAY736 to ibrutinib can deepen responses and increase the complete response rate.

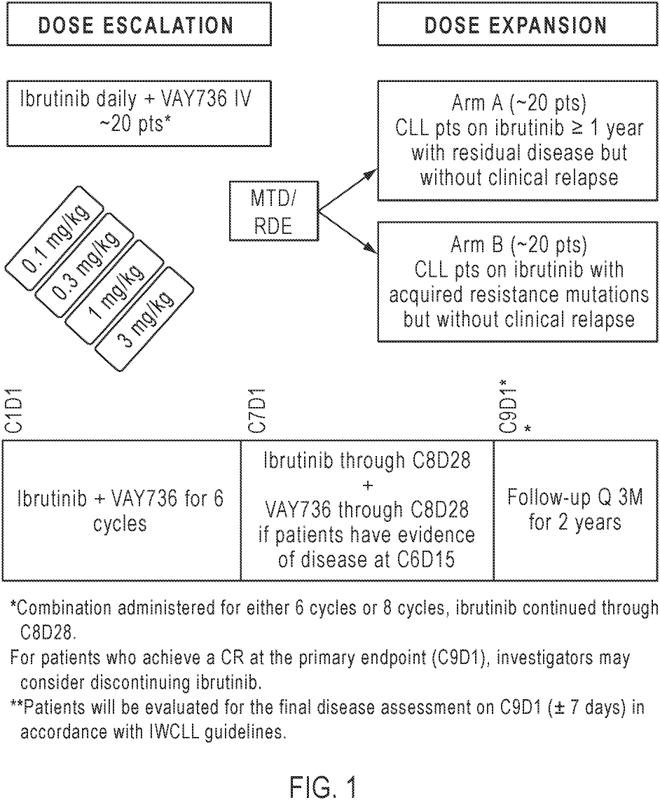

Patients will receive the combination of VAY736 and ibrutinib for 6 cycles. Starting with C7D1 (i.e. Cycle 7 Day 1), VAY736 will be discontinued if the patient has achieved a CR per IWCLL response criteria with no evidence of disease per radiological assessment and normal blood counts at C6D15. Ibrutinib will be administered per protocol (420 mg orally administered daily) for an additional two cycles (through C8D28). If a patient has not achieved a CR per IWCLL response criteria at C6D15, VAY736 and ibrutinib will be continued for 2 additional cycles (Cycle 7 and Cycle 8). At C9D1 (±7 days), patients will have their final disease assessment (including MRD evaluation) of the study. All patients who have remained on study, including those who have discontinued treatment due to reasons other than disease progression, will be assessed at this time point. For patients who achieve a complete response at this assessment, investigators may consider discontinuing ibrutinib in order to follow patients for durability of response and minimize intolerance and toxicity associated with continuous ibrutinib therapy while maximizing patient quality of life. See FIG. 1.

The study will end when all patients have completed the treatment period, safety period and two-year efficacy follow-up or have been lost to follow-up, discontinued the study for any reason, or the study is terminated early.

As of Mar. 24, 2020, in the ongoing CVAY736Y2102 clinical trial, a total of 15 patients with refractory, relapsing CLL have received VAY736 at various doses, including 0.3 mg/kg, 1 mg/kg, 3 mg/kg and 9 mg/kg IV every 2 weeks. No DLTs have been observed. Of the 9 patients who reached the primary endpoint (C9D1), three treated in the 0.3 mg/kg cohort, two treated in the 1.0 mg/kg cohort and 4 treated in the 3.0 mg/kg cohort, four patients have achieved a complete response.

### 2.1.2. Patient Population

The study will enroll patients with CLL who are currently taking ibrutinib therapy following relapse from another approved therapy AND have either failed to achieve a CR after >1 year of ibrutinib treatment OR who have developed a resistance mutation to ibrutinib without clinical relapse at any time during treatment. The dose expansion part of the study will also include patients who have received ibrutinib either alone or in combination (or have received ibrutinib continuously with multiple sequential combination partners) as first-line therapy and have either failed to achieve a complete response after 1 year of therapy or have developed a resistance mutation to ibrutinib. Patients must be taking and tolerating ibrutinib at time of enrollment with no limits to their continued ibrutinib use.

The investigator or designee must ensure that only patients who meet all the following inclusion and none of the exclusion criteria are offered treatment in the study.

### 2.1.3. Inclusion Criteria

Patients eligible for inclusion in this study have to meet all of the following criteria:

Patient must meet the following laboratory values at the screening visit unless cytopenias are related to CLL:

1- Diagnosis of chronic lymphocytic leukemia (CLL) meeting criteria established in the World Health Organization (WHO) classification of hematologic disorders or International Workshop on Chronic Lymphocytic Leukemia (IWCLL) (Hallek, M, Cheson, BD, Catovsky, D, et al. (2018 iwCLL guidelines for diagnosis, indications for treatment, response assessment, and supportive management of CLL. Blood,131, 2745-2760). Variant immunophenotype and prolymphocytic morphology change after diagnosis of CLL is allowed.

2- Age ≥ 18 years

3- Women of child-bearing potential agree to avoid pregnancy as per the ibrutinib package insert.

4- Male patients agree to avoid fathering a child as per the ibrutinib package insert.

5- Dose escalation:
a. On ibrutinib > 1 year following relapse from another approved therapy without achieving a complete response OR
b. On ibrutinib following relapse with another approved therapy and the presence of a known ibrutinib resistance mutation (BTK or PLCγ2) at ≥1 variant allele frequency OR <1% with two separate measurements at least 4 weeks apart with increasing variant allele frequency.

6- Dose expansion:
a. Arm A: On ibrutinib > 1 year following relapse from another approved therapy without achieving a complete response and patients who have received ibrutinib either alone or in combination (or have received ibrutinib continuously with multiple sequential combination partners) as first-line therapy and have failed to achieve a complete response after 1 year.
b. Arm B: On ibrutinib following relapse with another approved therapy and patients who have received ibrutinib either alone or in combination (or have received ibrutinib continuously with multiple sequential combination partners) as first-line therapy and the presence of a known ibrutinib resistance mutation at ≥1% variant allele frequency OR <1% with two separate measurements at least 4 weeks apart with increasing variant allele frequency

7- Ibrutinib dose:
Escalation: Patients must be receiving 420 mg ibrutinib
Expansion: Patients may be on a dose of ibrutinib lower than 420 mg. Any dose must have been stable for 2 months prior to the start of study treatment.

8- Absolute Neutrophil Count ≥ 750 cells/µL (0.75 x 10⁹/L) independent of growth factor support within 7 days of the first dose of VAY736.

9- Platelets ≥25 × 10⁹/L without transfusion support within 7 days of the first dose of study drug. Patients with transfusion dependent thrombocytopenia are excluded.

10- Hemoglobin (Hgb) ≥8 g/dL without transfusion support within 7 days prior to the first dose of VAY736.

11- Creatinine Clearance ≥ 30 mL/min using Cockcroft-Gault formula (or similar institutional standard) or creatinine < 2× ULN.

12- Total bilirubin ≤1.5 × ULN (For patients with Gilbert’s Syndrome: total bilirubin < 3.0 x ULN with direct bilirubin < 1.5 × ULN).

13- Aspartate transaminase (AST) ≤ 3.0 × ULN.

14- Alanine transaminase (ALT) ≤ 3.0 × ULN.

15- Eastern Cooperative Oncology Group (ECOG) performance status 0-2.

16- Written informed consent must be obtained prior to any screening procedures.

17.Patients with relapsed disease after prior allogeneic stem cell transplant (myeloablative or nonmyeloablative) will be eligible if they meet all other inclusion criteria and:
a. Do not have active (chronic or acute) GVHD and no immunosuppression
b. Are more than 6 months from transplant

### 2.1.4. Exclusion Criteria

1- History of transformation to aggressive disease histology (Large cell lymphoma) within 2 years prior to enrollment.

2- Malignant disease, other than that being treated in this study. Exceptions to this exclusion include the following: malignancies that were treated curatively and have not recurred within 2 years prior to study entry; completely resected basal cell and squamous cell skin cancers, superficial bladder cancer, and completely resected carcinoma in situ of any type.

3- Received chemotherapy, anticancer antibodies, investigational drug, or any drug used in combination with ibrutinib as first line therapy or as a sequential combination partner, within 30 days prior to the first dose of study drug.

4- Non-palliative radiotherapy within 2 weeks prior to the first dose of study drug. Palliative radiotherapy to a limited field, such as for the treatment of bone pain or focally painful tumor mass is allowed. To allow for assessment of response to treatment, patients must have remained measurable disease that has not been irradiated

5- History of hypersensitivity to any of the study drugs or to drugs of similar chemical classes (e.g., mAb of IgG1 class)

6- Receipt of attenuated vaccine within a 2 week period before VAY736 treatment

7- All acute toxic effects of any prior antitumor therapy (including ibrutinib) resolved to ≤ Grade 1 before study enrollment (with the exception of alopecia, grade 2 neurotoxicity, or grade 2 or 3 bone marrow parameters)

8- Presence of active CNS disease

9- Known history of HIV infection

10- Active hepatitis C infection defined by a positive RNA PCR test and/or hepatitis B infection defined as:
Positive serology for hepatitis B surface antigen (HBsAg)
Positive serology for hepatitis B core antibody (HBcAb), except if all 3 following criteria are met:
   i) HBV DNA negative
   ii) prophylactic treatment (with nucleos/tide) initiated latest on day 1 and continued until 12 months after last treatment
   iii) hepatitis B monitoring is implemented: HBsAg (and HBV DNA) tested every 4 weeks until the end of prophylactic treatment.

11- Active, uncontrolled autoimmune cytopenias (including autoimmune hemolytic anemia or immune thrombocytopenia)

12- Current treatment with medications or consuming foods that are strong/moderate inhibitors or strong inducers of CYP3A that cannot be discontinued at least one week prior to the start of treatment.

13- Risk factors for Torsades de Pointes (TdP) including uncorrected hypokalemia or hypomagnesemia, history of cardiac failure, or history of clinically significant/symptomatic bradycardia

14- Impaired cardiac function or clinically significant cardiac disease, including any of the following:
Clinically significant and/or uncontrolled heart disease such as congestive heart failure requiring treatment (NYHA Grade ≥ 2), uncontrolled hypertension or clinically significant arrhythmia
Acute myocardial infarction or unstable angina pectoris < 3 months prior to study entry 15- Patients with impaired hepatic function as defined by Childs-Pugh class B or C.

16- History of stroke or intracranial hemorrhage within 6 months prior to start of study drug

17- Evidence of active ongoing systemic bacterial, mycobacterial, fungal, or viral infection at the time of study enrollment. Note: Subjects with localized fungal infections of skin or nails are eligible. Subjects may be receiving prophylactic antiviral or antibacterial therapies at the discretion of the investigator.

18- Impairment of gastrointestinal function or gastrointestinal disease that may significantly alter the absorption of study drugs, with the exception of prior gastrectomy (e.g., ulcerative disease, uncontrolled nausea, vomiting, diarrhea, malabsorption syndrome)

19- Unable or unwilling to swallow the oral drug as per dosing schedule

20- Two weeks since major surgery treatment (mediastinoscopy, insertion of a central venous access device and insertion of a feeding tube are not considered major surgery)

21- Current use of therapeutic doses of warfarin sodium or any other Coumadin-derivative anticoagulants.

22- Ongoing immunosuppressive therapy, including systemic corticosteroids for treatment of CLL. Note: Subjects may use topical or inhaled corticosteroids as therapy for comorbid conditions and low-dose systemic corticosteroids (≤25 mg/day of prednisone or equivalent) for endocrine or rheumatologic conditions. During study participation, subjects may receive systemic or other corticosteroids as pretreatment for VAY736 infusions or as needed for treatment-emergent comorbid conditions.

23- Life-threatening illness, medical condition or organ system dysfunction which, in the investigator’s opinion, could compromise the subject’s safety, or put the study outcomes at undue risk.

24- Women of child-bearing potential, defined as all women physiologically capable of becoming pregnant, unless they are using highly effective methods of contraception during dosing of VAY736 and for 4 months after stopping VAY736. Highly effective contraception methods include:
Total abstinence (when this is in line with the preferred and usual lifestyle of the subject. Periodic abstinence (e.g., calendar, ovulation, symptothermal, post-ovulation methods) and withdrawal are not acceptable methods of contraception
Female sterilization (have had surgical bilateral oophorectomy with or without hysterectomy), total hysterectomy, or tubal ligation at least six weeks before taking study treatment. In case of oophorectomy alone, only when the reproductive status of the woman has been confirmed by follow up hormone level assessment
Male sterilization (at least 6 months prior to screening). The vasectomized male partner should be the sole partner for that subject
Use of oral, injected or implanted hormonal methods of contraception or placement of an intrauterine device (IUD) or intrauterine system (IUS), or other forms of hormonal contraception that have comparable efficacy (failure rate <1%), for example hormone vaginal ring or transdermal hormone contraception.

In case of use of oral contraception women should have been stable on the same pill for a minimum of 3 months before taking study treatment.

Women are considered post-menopausal and not of child bearing potential if they have had 12 months of natural (spontaneous) amenorrhea with an appropriate clinical profile (i.e. age appropriate, history of vasomotor symptoms) or have had surgical bilateral oophorectomy (with or without hysterectomy), total hysterectomy, or tubal ligation at least six weeks ago. In the case of oophorectomy alone, only when the reproductive status of the woman has been confirmed by follow up hormone level assessment is she considered not of child bearing potential.

### 2.1.5. Dosing Regimen

The dosing cycle is 28 days. Patients will receive ibrutinib once daily continuously and VAY736 by i.v. (intravenous) once every 2 weeks (Days 1 and 15). During the dose escalation part of the study, the dose of ibrutinib will be 420 mg, and will not be escalated. During the dose expansion part of the study, ibrutinib will be continued at the same dose schedule as tolerated before study enrollment, see exclusion criteria above for additional information on dose level.

A Q4W dosing schedule may also be evaluated.

TABLE 3

Dose and treatment schedule.

| | | | |
|---|---|---|---|
| Study treatments | Pharmaceutical form and route of administration | Dose | Frequency and/or Regimen |
| VAY736 | Powder for solution for infusion | As assigned | Once every 2 weeks or every 4 weeks, as assigned |
| Ibrutinib | Solid dose (tablet or capsule) for oral use | Dose escalation: 420 mg Dose expansion: 420 mg or highest tolerated dose | Daily (28 day cycles) |

### 2.1.6. Treatment Duration

Patients will receive VAY736 and ibrutinib in combination for up to a total of six cycles. Patients will continue on VAY736 and ibrutinib therapy for Cycle 7 and Cycle 8 if the patient has evidence of disease at the radiological assessment or abnormal blood counts defined by IWCLL response criteria at C6D15. Starting with C7D1, VAY736 will be discontinued if the patient has no evidence of disease in radiological assessment and normal blood at C6D15 and ibrutinib will be administered for the next two cycles. Final response assessment (including MRD) is conducted at C9D1. Further treatment with ibrutinib is dependent upon the outcome of the assessment that is conducted at C9D1 (see Section 2.1.9).

### 2.1.7. Starting Dose Rationale for VAY736

The starting dose of VAY736 is 0.3 mg/kg i.v. Q2W on a 28-day cycle. The selection of the starting dose for this study in CLL patients was determined based on the available PK/PD modeling developed for a single agent study in RA. Differences between RA and CLL patients were considered for the simulations, including higher B cell baseline due to the presence of leukemia, lower BAFF density (Defoiche J, Debacq C, Asquith B, et al. (2008) Reduction of B Cell Turnover in Chronic Lymphocytic Leukaemia. Brit J Haematology, 143, 240-247; Mihalcik SA, Tschumper RC, & Jelinek DF. (2010) Transcriptional and Post-Transcriptional Mechanisms of BAFF-receptor dysregulations in Human B Lineage Malignancies. Cell Cycle 9:24, 4884-4892), and potentially compromised ADCC effect due to long-term pre-treatment of ibrutinib (Kohrt HE, Sagiv-Barfi I, Rafiq S, et al. (2014) Ibrutinib antagonizes rituximab-dependent NK cell-mediated cytotoxicity. Blood, 123, 1957-1960; Ysebaert L, Klein C, & Quillet-Mary A. (2014). Ibrutinib Exposure and B-Cell Depletion Induced By Anti-CD20 Monoclonal Antibodies Rituximab and Obinutuzumab: Is There a Rationale for Combination Studies? Blood, 124(21), 1980). Simulations suggest that VAY736 at 0.3 mg/kg i.v. Q2W can provide BAFF receptor occupancy and B cell depletion greater than 90% over the entire 14-day dosing interval. Q4W dosing may be considered based on emerging clinical data. The DLT data available from VAY736 Q2W will be used to derive a MAP prior distribution for the Q4W dosing schedule and separate BLRM will be set up for Q4W schedule. The starting dose for Q4W will not exceed the highest tolerated dose evaluated using the Q2W schedule and satisfies the EWOC criteria for Q4W.

### 2.1.8. Provisional Dose Levels

Table 4 describes the starting dose and the dose levels of VAY736 that may be evaluated during this trial. Ibrutinib will be administered at 420 mg daily during dose escalation. During the dose expansion part of the study, ibrutinib will be continued at the same dose schedule as tolerated before study enrollment.

TABLE 4

VAY736 provisional dose levels

| Dose level | Proposed dose of VAY736* |
|---|---|
| -1^{∗∗} | 0.1 mg/kg |
| 1 | 0.3 mg/kg |
| 2 | 1 mg/kg |
| 3^{∗∗∗} | 3 mg/kg |

^{∗}Proposed dose regardless of whether Q2W or Q4W regimen is used. It is possible for additional and/or intermediate dose levels to be added during the course of the study. Cohorts may be added at any dose level below the MTD in order to better understand safety, PK, or PD.
^{∗∗}Dose level -1 represents treatment doses for patients requiring a dose reduction from the starting dose level. No dose reduction below dose level -1 is permitted for this study.
^{∗∗∗}If clinically indicated, dose levels higher than 3.0 mg/kg may be investigated.

### 2.1.9. Treatment Period

The study treatment period for each patient begins when the patient receives the first dose of VAY736 in combination with ibrutinib and ends at C9D1. Patients will receive the combination of VAY736 and ibrutinib for six cycles. Patients will continue VAY736 for 2 additional cycles in combination with ibrutinib if there is evidence of disease at the radiological assessment or abnormal blood counts defined by IWCLL response criteria at C6D15. For the purpose of scheduling and evaluations, a treatment cycle is 28 days. VAY736 will be discontinued at C7D1 if the patient shows a complete radiological response or normal blood counts at C6D15 and ibrutinib will continue for an additional 2 cycles. All patients will have final response assessment (including MRD assessment) at C9D1. Further treatment or follow-up will depend on status of response at C9D1. One of the following actions will be taken for patients:
Patients who experience disease progression at C9D1 (or any time prior to C9D1) will discontinue treatment and will have required end of treatment assessments conducted.
Patients whose disease is assessed as stable disease, partial response, at C9D1 may continue ibrutinib and will be followed every 3 months for two years for TTP assessments, with the exception of CT scans which will be performed every 6 months unless the patient experiences disease progression or the administration of a new therapy. If the patient continues to receive ibrutinib past C9D1, the details of administration will be recorded on the Antineoplastic therapies since discontinuation eCRF.
For patients whose disease is assessed as CR at C9D1, investigators may consider discontinuing ibrutinib, and be followed every 3 months for two years for TTP assessments, with the exception of CT scans which will be performed every 6 months unless the patient experiences disease progression or the administration of a new therapy. Although investigators may consider discontinuing ibrutinib if patients have a CR at C9D1, if the patient continues to receive ibrutinib past C9D1, the details of administration will be recorded on the Antineoplastic therapies since discontinuation eCRF.

Patients who discontinue one of the combination drugs (ibrutinib or VAY736) prior to six cycles of treatment for reasons other than disease progression may continue the other drug until the end of treatment period (VAY736 until C6D28 or ibrutinib until C8D28). All patients regardless of study treatment duration, who have not progressed, will have a final disease assessment at C9D1 (end of treatment period).

### 2.1.10. Efficacy Assessments

Efficacy will be evaluated as per the IWCLL Guidelines (Table 5 and 6).

Tumor assessments will be performed at screening. All screening tumor assessments should be performed as closely as possible to the start of treatment (preferably within 7 days) and never more than 28 days before the start of treatment. On-treatment radiological examinations and MRD assessments have a +/- 7 day window.

Clinical suspicion of disease progression at any time will require a disease evaluation promptly, rather than waiting for the next scheduled tumor assessment. In case of an unscheduled or delayed disease evaluation for any reason, subsequent tumor assessments should be performed according to the originally planned schedule unless a scan has been performed within 28 days.

All patients discontinuing from the study for progressive disease must have their disease progression documented.

Chest, abdomen and pelvis CT scans are required for all subjects at screening. If clinically indicated, neck CT scans should also be acquired at screening. Post baseline scans should only be performed in these anatomical regions that demonstrated disease at baseline. In case of clinical complete response, confirmation scans of chest, abdomen and pelvis are required and neck if appropriate.

CT scans should be acquired with intravenous (i.v.) contrast. If a patient is known to have a medical contraindication to CT i.v. contrast agent or develops a contraindication during the study, a CT scan without contrast should be acquired. If inguinal and/or femoral nodes are present, every effort should be made to ensure that pelvis CT scans cover both inguinal areas in their entirety.

Magnetic resonance imaging (MRI) will be allowed only in those cases when CT scans cannot be performed. Each lesion that is measured at baseline/screening must be measured by the same method throughout the study so that the comparison is consistent. For complete details, refer to Table 5 and 6.

For patients who discontinue treatment for reasons other than documented disease progression, death, lost to follow-up, or withdrawal of consent, tumor assessments must continue to be performed as appropriate for the dosing regimen of VAY736, until documented disease progression, death, lost to follow-up, or withdrawal of consent.

Minimal residual Disease (MRD) will be evaluated. MRD in blood and bone marrow will be assessed by central multi-parameter flow cytometry. Assessments will be performed at baseline, and during the treatment period until disease progression. MRD negativity will be defined based on the detection of CLL immunophenotype comprising a core panel of 6 markers (i.e, CD19, CD20, CD5, CD43, CD79b, and CD81) (Hallek, M, Cheson, BD, Catovsky, D, et al (2018). iwCLL guidelines for diagnosis, indications for treatment, response assessment, and supportive management of CLL. Blood, 131, 2745-2760). As such, patients will be defined as having undetectable MRD (MRD-negative) if they have blood or marrow with <1 CLL cell per 10, 000 leukocytes.

Tables 5 and 6 refer to Hallek, M, Cheson, BD, Catovsky, D, et al (2018): iwCLL guidelines for diagnosis, indications for treatment, response assessment, and supportive management of CLL. Blood, 131, 2745-2760.

TABLE 5

Response definition after treatment for patients with CLL.

| Group | Parameter | CR | PR | PD | SD |
|---|---|---|---|---|---|
| A | Lymph nodes | None ≥1.5 cm | Decrease ≥50% (from baseline)* | Increase ≥50% from baseline or from response | Change of -49% to +49% |
| | Liver and/or spleen size† | Spleen size <13 cm; liver size normal | Decrease ≥50% (from baseline) | Increase ≥50% from baseline or from response | Change of -49% to +49% |
| | Constitutional symptoms | None | Any | Any | Any |
| | Circulating lymphocyte count | Normal | Decrease ≥50% from baseline | Increase ≥50% over baseline | Change of -49% to +49% |
| B | Platelet count | ≥ 100 ×10⁹/L | ≥100 × 10⁹/L or increase ≥50% over baseline | Decrease of ≥50% from baseline secondary to CLL | Change of -49 to +49% |
| | Hemoglobin | ≥11.0 g/dL (untransfused and without erythropoietin) | ≥11 g/dL or increase ≥50% over baseline | Decrease of ≥2 g/dL from baseline secondary to CLL | Increase <11.0 g/dL or <50% over baseline, or decrease <2 g/dL |
| | Marrow | Normocellular, no CLL cells, no B-lymphoid nodules | Presence of CLL cells, or of B-lymphoid nodules, or not done | Increase of CLL cells by ≥50% on successive biopsies | No change in marrow infiltrate |

*Sum of the products of 6 or fewer lymph nodes (as evaluated by CT scans and physical examination in clinical trials or by physical examination in general practice).
†Spleen size is considered normal if <13 cm. There is not firmly established international consensus of the size of a normal liver; therefore, liver size should be evaluated by imaging and manual palpation in clinical trials and be recorded according to the definition used in a study protocol.
CR, complete remission (all of the criteria have to be met); PD, progressive disease (at least 1 of the criteria of group A or group B has to be met); PR, partial remission (for a PR, at least 2 of the parameters of group A and 1 parameter of group B need to improve if previously abnormal; if only 1 parameter of both groups A and B is abnormal before therapy, only 1 needs to improve); SD, stable disease (all of the criteria have to be met; constitutional symptoms alone do not define PD).

TABLE 6

Grading scale for hematologic toxicity in CLL studies.

| Grade^{∗} | Decrease in platelets^{†} or Hb^{‡} (nadir) from pretreatment value, % | Absolute neutrophil count/µL^{§} (nadir) |
|---|---|---|
| 0 | No change to 10% | ≥ 2000 |
| 1 | 11%-24% | ≥ 1500 and < 2000 |
| 2 | 25%-49% | ≥ 1000 and < 1500 |
| 3 | 50%-74% | ≥ 500 and < 1000 |
| 4 | ≥ 75% | < 500 |

^{∗} Grades: 1, mild; 2, moderate; 3, severe; 4, life-threatening; 5, fatal. Death occurring as a result of toxicity at any level of decrease from pretreatment will be recorded as grade 5. † Platelet counts must be below normal levels for grades 1 to 4. If, at any level of decrease, the platelet count is < 20 × 10⁹/L (20000/µL), this will be considered grade 4 toxicity, unless a severe or life-threatening decrease in the initial platelet count (eg, 20 × 10⁹/L [20000/uL]) was present pretreatment, in which case the patient is not evaluable for toxicity referable to platelet counts.
‡ Hb levels must be below normal levels for grades 1 to 4. Baseline and subsequent Hb determinations must be performed before any given transfusions. The use of erythropoietin is irrelevant for the grading of toxicity but should be documented.
§ If the absolute neutrophil count (ANC) reaches < 1 × 10⁹/L (1000/µL), it should be judged to be grade 3 toxicity. Other decreases in the white blood cell count, or in circulating neutrophils, are not to be considered because a decrease in the white blood cell count is a desired therapeutic endpoint. A gradual decrease in granulocytes is not a reliable index in CLL for stepwise grading of toxicity. If the ANC was < 1 × 10⁹/L (1000/uL) before therapy, the patient is not evaluable for toxicity referable to the ANC. The use of growth factors such as G-CSF is not relevant to the grading of toxicity, but should be documented.

### 2.1.11. Primary Objective

The primary objective is to characterize the safety and tolerability of the combination of VAY736 with ibrutinib and to determine the MTD/RD for expansion.

### 2.1.12. Secondary Objective

Secondary objectives are described in Table 7.

TABLE 7

Objectives and related endpoints

| Objective | Endpoint |
|---|---|
| Primary | |
| To determine the MTD and/or RD of the combination of VAY736 with ibrutinib | Safety Escalation only: • Incidence of DLTs in cycle 1 (28 days) Escalation and expansion: • Incidence and severity of AEs and SAEs, including changes in laboratory parameters and vital signs |
| To characterize the safety and tolerability of the combination of VAY736 and ibrutinib | |
| | Tolerability Dose interruptions, reduction, and dose intensity |
| Secondary | |
| To assess any preliminary antitumor activity of the combination | Rate of patients with CR as assessed by investigators per IWCLL at cycle 9 day 1 |
| | Overall response rate (ORR) assessed by investigators per IWCLL criteria and Time To Progression (TTP) |
| | Clearance of ibrutinib resistance mutations (BTKC481 and/or PLCy2 hotspot), defined as less than 1% mutation bearing alleles (Arm B only). |
| To characterize the PK of VAY736 and ibrutinib when used in combination therapy | Plasma concentration of VAY736 and ibrutinib, and derived parameters |
| To assess immunogenicity (IG) following one or more intravenous infusions of VAY736 | Presence of anti-VAY736 antibodies |
| Exploratory | |
| To assess markers that may correlate with prediction of response and/or resistance | B cell depletion, NK-cell number and subsets, biomarkers related to BAFF signaling, expression of BAFF-R in leukemic clones |
| To characterize the pharmacodynamic profiles of VAY736 in combination with ibrutinib | Concentration-time profiles, B cell depletion, BAFF Receptor Occupancy (RO) and soluble BAFF (sBAFF) when VAY736 is given with ibrutinib |
| To explore the possible relationship of baseline cytogenetic risk factors with anti-tumor activity | Complex karyotype (yes/no), del17p, del11q, trisomy12, del13q, IGHV% (mutated/un-mutated), clinical response |
| To assess any exploratory antitumor activity of the combination | Rate of patients with MRD negativity at cycle 9 day 1 |

All efficacy analyses are based on FAS.

Rate of CR at C9 for expansion arm A and arm B: The proportion of patients with CR, assessed by investigators per IWCLL criteria (see Table 5 and 6 for details) at C9 will be provided. The rate of CR at C9 is the primary endpoint for the evaluation of anti-tumor activity and will be analyzed for each expansion arm using a Bayesian modeling approach.

A minimally informative beta distribution is used as prior distribution with parameters a=0.25 and b=1. This assumes a priori response rate of 20%.

Posterior summaries for CR rate (posterior mean, including 90% credible intervals and the posterior probability that the true CR rate falls in the activity intervals defined below) will be provided:

Posterior probability of response rate intervals:
[0, 20%) - clinically not meaningful
[20%, 40%) - moderate clinical benefit
[40%, 100%] - superior clinical benefit

In addition, exact confidence interval (90% CI) also will be provided.

Clearance of ibrutinib resistance mutation for expansion arm B is defined as less than 1% mutation bearing alleles (BTKC481 and/or PLCγ2) during treatment. The proportion of patients with negative mutation will be provided along with corresponding 90% exact confidence interval (CI).

Overall response rate (ORR) is defined as best overall response (BOR) of complete response (CR) or partial response (PR), assessed by investigators per IWCLL criteria (see Table 5 and 6 for details). ORR for each expansion arm along with corresponding 90% exact confidence interval (CI) will be provided.

Time to progression (TTP) is the time from start of treatment to the date of event which is defined as the first documented progression or death due to underlying cancer. If a patient has not had an event, time to progression is censored at the date of last adequate disease assessment.

TTP will be described using Kaplan-Meier methods and appropriate summary statistics.

### 2.2. Results

A total of 15 patients (median age: 65 years; ECOG PS 0: 93%) were treated by the data cutoff (Jun. 9, 2020). Overall, 11 patients completed and 3 discontinued combination treatment (primarily due to disease progression); 1 patient remains on treatment. Most patients (73%) had an ibrutinib resistance mutation at baseline (mainly [82%] BTKC481) and 33% had received ≥4 prior regimens (median: 3, range: 1-5); median duration of prior ibrutinib was 4.1 y (range: 0.2-8.3). Baseline cytogenetics were (not mutually exclusive): 27% del(17)(p13.1), 80% unmutated IGHV, 80% stimulated complex karyotypes (≥3 abnormalities), 60% del(13)(q14), and 7% +12.

No dose-limiting toxicities have been observed and the MTD has not been reached. A total of 14 (93%) patients experienced an AE regardless of cause. Four (27%) patients experienced AEs of grade ≥3, including decreased neutrophil count (n=3), hypophosphatemia (n=2), decreased white blood cell count, leukocytosis, increased lymphocyte count, hypertension, hypokalemia, and hypomagnesemia (n=1 each).

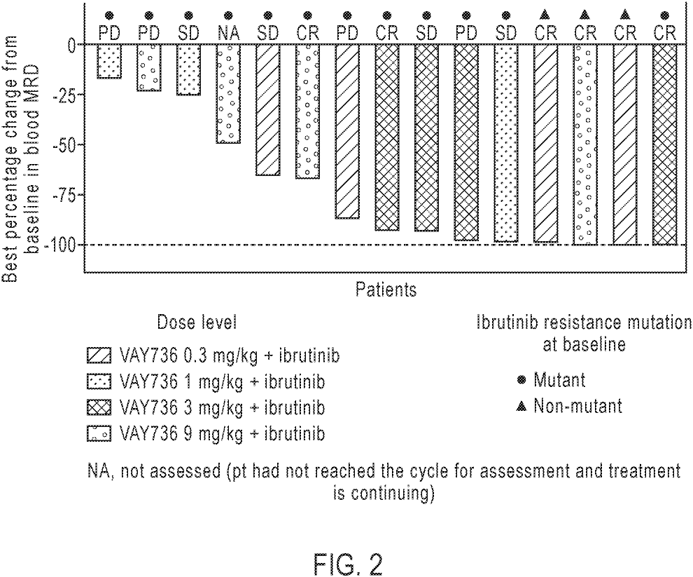

The overall response at C9D1 was CR in 6 (40%) patients, SD in 4 (27%) patients, PD in 4 (27%) patients, and not assessed in 1 (7%) patient (still on treatment). The mean baseline CLL cells in bone marrow for the CR, SD, and PD groups were 27% (range: 0.8-60.6%), 13% (range: 2.5-27%), and 66% (range: 47-77.9%). Three (20%) patients with CR achieved MRD-negativity and were able to discontinue CLL-directed therapy including ibrutinib; they remained in CR for 1-16 months after ibrutinib discontinuation. The median percentage change from baseline in blood MRD was -92.8% (range: -100%; -16.7%; FIG. 2) and in bone marrow MRD was -89.6% (range: -100%; -32.6%). Of the patients who had baseline ibrutinib-resistance mutations and C9D1 assessments, 1 patient (1/6) tested negative for ibrutinib resistance mutations at C9D1. None of the patients who were ibrutinib-resistance mutation negative at baseline (4/4) developed mutations by C9D1.

VAY736 concentration increased with dose, accumulated after repeated dosing in combination with ibrutinib, and achieved linear PK at 3 mg/kg or above. Tissue receptor occupancy was >99% for VAY736 doses of 3 mg/kg or above. Free BAFF was accumulated to steady state with no dose relationship.

### 2.3. Conclusions

VAY736 + ibrutinib had an acceptable safety profile and demonstrated promising preliminary activity in patients with R/R CLL on ibrutinib, providing clinical evidence of a potential to discontinue ibrutinib by VAY736 add-on therapy. Further investigation of this combination including in patients on first line ibrutinib and other ibrutinib combinations is ongoing.

1-48. (canceled)

49. A method of treating a subject having a B cell malignancy, comprising administering therapeutically effective dose of an anti-BAFFR antibody or a binding fragment thereof to the subject.

50. The method according to claim 49, wherein the anti-BAFFR antibody or binding fragment thereof comprises CDR-H1, CDR-H2, and CDR-H3 having the amino acid sequences of SEQ ID NO:3, SEQ ID NO:4, and SEQ ID NO:5, and CDR-L1, CDR-L2, and CDR-L3 having the amino acid sequences of SEQ ID NO:6, SEQ ID NO:7, and SEQ ID NO:8, respectively.

51. The method according to claim 49, wherein the anti-BAFFR antibody or binding fragment thereof comprises a heavy chain variable region having the amino acid sequence of SEQ ID NO: 1 and a light chain variable region having the amino acid sequence of SEQ ID NO: 2.

52. The method according to claim 49, wherein the anti-BAFFR antibody or binding fragment thereof is ianalumab or a binding fragment thereof.

53. The method according to claim 49, wherein the anti-BAFFR antibody or binding fragment thereof is to be administered at a dose of 0.1 mg/kg to 20 mg/kg, 1 mg/kg to 10 mg/kg, 5 mg/kg to 15 mg/kg, or 10 mg/kg to 20 mg/kg.

54. The method according to claim 53, wherein the anti-BAFFR antibody or binding fragment thereof is to be administered at a dose of 1 mg/kg, 3 mg/kg, 6 mg/kg, 9 mg/kg, 12 mg/kg, or 20 mg/kg.

55. The method according to claim 49, wherein the anti-BAFFR antibody or binding fragment thereof is to be administered to a subject in need thereof once every two weeks (+/- 3 days), once every week (+/- 3 days), or once every 4 weeks (+/- 3 days).

56. The method according to claim 49, wherein the anti-BAFFR antibody or binding fragment thereof is to be administered at a dose of 3 mg/kg, once every two weeks (+/- 3 days).

57. The method according to claim 49, wherein the anti-BAFFR antibody or binding fragment thereof is to be administered at a dose of 9 mg/kg, once every four weeks (+/- 3 days).

58. The method according to claim 49, wherein the anti-BAFFR antibody or binding fragment thereof is to be administered intravenously to a subject in need thereof.

59. The method according to claim 49, wherein the anti-BAFFR antibody or binding fragment thereof is to be administered as monotherapy for the B cell malignancy.

60. The method according to claim 49, wherein the anti-BAFFR antibody or binding fragment thereof is to be administered in combination with one or more additional agents.

61. The method according to claim 60, wherein the one or more additional agents comprise an immunomodulatory imide drug (IMiD).

62. The method according to claim 61, wherein the IMiD is lenalidomide or a pharmaceutically acceptable salt thereof, thalidomide or a pharmaceutically acceptable salt thereof, pomalidomide or a pharmaceutically acceptable salt thereof, or iberdomide or a pharmaceutically acceptable salt thereof.

63. The method according to claim 62, wherein the IMiD is lenalidomide or a pharmaceutically acceptable salt thereof.

64. The method according to claim 63, wherein the lenalidomide or a pharmaceutically acceptable salt thereof is to be administered at a dose of 2.5 mg to 25 mg.

65. The method according to claim 64, wherein the lenalidomide or a pharmaceutically acceptable salt thereof is to be administered at a dose of 2.5 mg, 5 mg, 15 mg, 20 mg, or 25 mg.

66. The method according to any one of claims 63 to 65, wherein the lenalidomide or a pharmaceutically acceptable salt thereof is to be administered to a subject in need thereof once a day.

67. The method according to claim 49, wherein the B cell malignancy is a plasma cell dyscrasia, acute leukemia, B cell acute lymphocytic leukemia (B-ALL), non-Hodgkin’s lymphoma (NHL), chronic lymphocytic leukemia (CLL)/small lymphocytic lymphoma (SLL), follicular lymphoma (FL), optionally wherein the FL is small cell FL or large cell FL, mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma), marginal zone lymphoma (MZL), extranodal marginal zone lymphoma (EMZL), nodal marginal zone B-cell lymphoma (NZML), or splenic marginal zone B-cell lymphoma (SMZL).

68. The method, or combination according to claim 67, wherein the subject has failed at least one prior line of standard of care therapy.

69. The method, or combination according to claim 68, wherein the at least one prior line of standard of care therapies comprise an anti-CD20 therapy.


SEQUENCE LISTING (15 sequences)

SEQ ID NO: 1 - protein, 124 aa, Artificial Sequence
  Synthetic polypeptide
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSS

SEQ ID NO: 2 - protein, 108 aa, Artificial Sequence
  Synthetic polypeptide
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIK

SEQ ID NO: 3 - protein, 12 aa, Artificial Sequence
  Synthetic polypeptide
GDSVSSNSAAWG

SEQ ID NO: 4 - protein, 18 aa, Artificial Sequence
  Synthetic polypeptide
RIYYRSKWYNSYAVSVKS

SEQ ID NO: 5 - protein, 12 aa, Artificial Sequence
  Synthetic polypeptide
YDWVPKIGVFDS

SEQ ID NO: 6 - protein, 12 aa, Artificial Sequence
  Synthetic polypeptide
RASQFISSSYLS

SEQ ID NO: 7 - protein, 7 aa, Artificial Sequence
  Synthetic polypeptide
GSSSRAT

SEQ ID NO: 8 - protein, 9 aa, Artificial Sequence
  Synthetic polypeptide
QQLYSSPMT

SEQ ID NO: 9 - protein, 454 aa, Artificial Sequence
  Synthetic polypeptide
QVQLQQSGPGLVKPSQTLSLTCAISGDSVSSNSAAWGWIRQSPGRGLEWLGRIYYRSKWYNSYAVSVKSRITINPDTSKNQFSLQLNSVTPEDTAVYYCARYDWVPKIGVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  Synthetic polypeptide
DIVLTQSPATLSLSPGERATLSCRASQFISSSYLSWYQQKPGQAPRLLIYGSSSRATGVPARFSGSGSGTDFTLTISSLEPEDFAVYYCQQLYSSPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 372 bp, Artificial Sequence
  Synthetic polynucleotide
caggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagc

SEQ ID NO: 12 - DNA, 324 bp, Artificial Sequence
  Synthetic polynucleotide
gatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaag

SEQ ID NO: 13 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 14 - DNA, 1419 bp, Artificial Sequence
  Synthetic polynucleotide
atggcctgggtgtggaccctgcccttcctgatggccgctgcccagtcagtgcaggcccaggtgcagctgcagcagagcggcccaggcctggtcaagccctctcagaccctgtcactgacctgcgccatttcaggcgacagcgtgagcagcaacagcgccgcctggggctggatcaggcagagccccggtaggggcctggaatggctgggcaggatctactacaggtccaagtggtacaacagctacgccgtgagcgtgaagagcaggatcaccatcaaccctgacaccagcaagaaccagttctcactgcagctcaacagcgtgacccccgaggacaccgccgtgtactactgcgccagatacgactgggtgcccaagatcggcgtgttcgacagctggggccagggcaccctggtgaccgtgtcaagcgccagcaccaagggccccagcgtgttccccctggcccccagcagcaagagcaccagcggcggcacagccgccctgggctgcctggtgaaggactacttccccgagcccgtgaccgtgtcctggaacagcggagccctgacctccggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagccccagagctgctgggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaggaatacaagtgcaaggtctccaacaaggccctgccagcccccatcgaaaagaccatcagcaaggccaagggccagccacgggagccccaggtgtacaccctgcccccctcccgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagagcctgagcctgtcccccggcaag

SEQ ID NO: 15 - DNA, 705 bp, Artificial Sequence
  Synthetic polynucleotide
atgagcgtgctgacccaggtgctggctctgctgctgctgtggctgaccggcaccagatgcgatatcgtgctgacacagagccccgccaccctgagcctgagcccaggcgagagggccaccctgtcctgcagggccagccagtttatcagcagcagctacctgtcctggtatcagcagaagcccggccaggcccctagactgctgatctacggcagctcctctcgggccaccggcgtgcccgccaggttcagcggcagcggctccggcaccgacttcaccctgacaatcagcagcctggagcccgaggacttcgccgtgtactactgccagcagctgtacagctcacccatgaccttcggccagggcaccaaggtggagatcaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc